(19) United States
(12) Patent Application Publication
(10) Pub. No.: US 2016/0143821 A1
(43) Pub. Date: May 26, 2016

(54) PACKAGED PRODUCT INCLUDING A CONTAINER WITH A CONSUMER MEDIUM AND A PROPELLANT MEDIUM STORED THEREIN

(71) Applicant: EVEREADY BATTERY COMPANY, INC, St. Louis, MO (US)

(72) Inventors: Pauley Chang, Milford, CT (US); Raymond Guimont, Guilford, CT (US)

(21) Appl. No.: 14/903,354

(22) PCT Filed: Jul. 9, 2014
(86) PCT No.: PCT/US2014/045895

§ 371 Date: Jan. 7, 2016

Related U.S. Application Data
(60) Provisional application No. 61/844,492, filed on Jul. 10, 2013.

(51) Int. Cl. A61K 8/04; A61Q 19/10; B65B 31/00; B65D 83/32; B65D 83/44; B65D 83/42; A61Q 9/02; A61Q 11/00

(52) U.S. Cl. CPC A61K 8/046; A61Q 9/02; A61Q 19/10; A61Q 11/00; B65D 83/32; B65D 83/44; B65D 83/42; B65B 31/003; A61K 2800/87

(57) ABSTRACT

A packaged product is provided that includes a container with a consumer medium and a propellant medium stored therein. The container is a non-barrier type aerosol container. The propellant medium includes a hydrocarbon agent. In some embodiments, the consumer medium is water-based and at least substantially free of solvents. In some embodiments, the propellant medium additionally includes an organoflourine agent and/or carbon dioxide.

### BACKGROUND

1. Technical Field

Aspects of the present invention relate to a packaged product, and more particularly relate to a packaged product that includes a container with a consumer medium and a propellant medium stored therein.

2. Background Information

It is known to provide a packaged product that includes a container with a consumer medium and a propellant medium stored therein. In some instances, the container is a barrier type aerosol container, and when the consumer medium and the propellant medium are dispensed from the container, they at least partially mix to form a consumer mixture. The barrier type aerosol container can be advantageous in that it can permit the consumer mixture to have one or more desirable characteristics. The barrier type aerosol container can be problematic, however, in that it can be relatively more expensive and relatively more prone to mechanical failure than other known types of containers, such as non-barrier type aerosol containers. Aspects of the present invention are directed to these and other problems.

### SUMMARY OF ASPECTS OF THE INVENTION

According to an aspect of the present invention, a packaged product is provided that includes a container with a consumer medium and a propellant medium stored therein. The container is a non-barrier type aerosol container. The propellant medium includes a hydrocarbon agent.

According to an additional or alternative aspect of the present invention, the container does not include a barrier that fluidly separates the consumer medium from the propellant medium.

According to an additional or alternative aspect of the present invention, the container includes a container reservoir and a container valve; the consumer medium and the propellant medium are stored within the container reservoir; the container valve is selectively actuatable between a closed position and an open position; the consumer medium and the propellant medium stored within the container reservoir cannot pass through the container valve when the container valve is in the closed position; and the consumer medium and the propellant medium stored within the container reservoir can pass through the container valve when the container valve is in the open position.

According to an additional or alternative aspect of the present invention, the volume of the reservoir is between approximately sixty (60) milliliters and three hundred fifty (350) milliliters.

According to an additional or alternative aspect of the present invention, the propellant medium provides a force that causes the consumer medium and the propellant medium to dispense from the container when the container valve is in the open position.

According to an additional or alternative aspect of the present invention, the consumer medium and the propellant medium are operable to mix with one another to form a consumer mixture as they dispense from the container.

According to an additional or alternative aspect of the present invention, the consumer medium is one of a shaving aid medium, a body wash medium, and a sunscreen medium.

According to an additional or alternative aspect of the present invention, the consumer medium is water-based.

According to an additional or alternative aspect of the present invention, the consumer medium is at least substantially free of solvents.

According to an additional or alternative aspect of the present invention, the hydrocarbon agent includes at least one of normal pentane, isopentane, normal butane, isobutane, and propane.

According to an additional or alternative aspect of the present invention, the propellant medium additionally include an organoflourine agent.

According to an additional or alternative aspect of the present invention, the organoflourine agent includes at least one of a difluoroethane compound, a trifluoropropene compound, a tetrafluoropropene compound, and a pentafluoropropane compound.

According to an additional or alternative aspect of the present invention, the propellant medium additionally includes carbon dioxide.

According to an additional or alternative aspect of the present invention, the propellant medium additionally includes air, and the carbon dioxide is provide in an amount that that is significantly greater than any amount of carbon dioxide already present in the air.

According to an additional or alternative aspect of the present invention, the propellant medium includes approximately twenty percent weight by volume of carbon dioxide.

According to an additional or alternative aspect of the present invention, a desired characteristic of the consumer mixture is achieved at least in part because the propellant medium has a particular vapor pressure at a particular temperature.

According to an additional or alternative aspect of the present invention, the propellant medium additionally includes carbon dioxide, and the desired characteristic of the consumer mixture is achieved at least in part because the carbon dioxide is included.

According to an additional or alternative aspect of the present invention, the desired characteristic is one of creaminess, cushion, lubricity, and hydration.

These and other aspects of the present invention will become apparent in light of the drawings and detailed description provided below.

### BRIEF DESCRIPTION OF THE DRAWINGS

FIG. 1 illustrates an embodiment of a packaged product.

FIG. 2 illustrates the hand of a consumer with an amount of the consumer mixture that was dispensed from the packaged product of FIG. 1.

### DETAILED DESCRIPTION

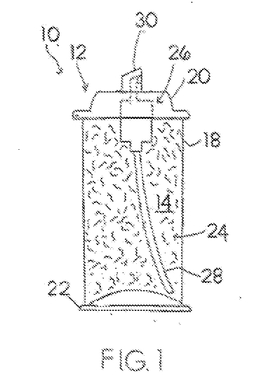

Referring to FIG. 1, the present disclosure describes embodiments of a packaged product 10 that includes a container 12 with a consumer medium 14 and a propellant medium 16 stored therein. The present disclosure describes aspects of the present invention with reference to the embodiments illustrated in the drawings; however, aspects of the present invention are not limited to the embodiments illustrated in the drawings.

The container 12 is a non-barrier type aerosol container. The term “aerosol container” is used herein to mean a container that includes, and is designed to include, an internal pressure source (e.g., a propellant medium) that is operable to dispense a consumer medium from the container. The term “barrier type aerosol container” is used herein to mean an aerosol container that includes a barrier (e.g., a piston, a bag, etc.) that fluidly separates a consumer medium from at least a portion of the internal pressure source. The term “non-barrier type aerosol container” is used herein to mean an aerosol container that does not include a barrier that fluidly separates a consumer medium from an internal pressure source.

The structure of the container 12 can vary. In the embodiment illustrated in FIG. 1, the container 12 is a non-barrier type aerosol container that includes a cylindrical container body 18; a container top 20 connected to a top end of the container body 18; a container bottom 22 connected to a bottom end of the container body 18; and a container reservoir 24 that is defined by the container body 18, the container top 20, and the container bottom 22. In this embodiment, the volume of the container reservoir 24 is approximately one hundred seventy five (175) milliliters (i.e., approximately six (6) ounces). In other embodiments, the volume of the reservoir 24 can be smaller or larger than one hundred seventy five (175) milliliters. The volume of the reservoir 24 can vary depending on the size of the container 12, but may typically be within the range, for example, of approximately sixty (60) milliliters to three hundred fifty (350) milliliters (i.e., approximately two (2) ounces to twelve (12) ounces). In the embodiment illustrated in FIG. 1, the container 12 additionally includes a container valve 26 that is disposed relative to the container top 20 and that is selectively actuatable between a closed position and an open position; the container 12 additionally includes a container dip tube 28 that is connected to an inlet portion of the container valve 26 and that extends into the container reservoir 24; and the container 12 additionally includes a container nozzle 30 that is connected to an outlet portion of the container valve 26. In this embodiment, when the container valve 26 is in the closed position, the consumer medium 14 and the propellant medium 16 stored within the container 12 cannot pass through the container valve 26; and when the container valve 26 is in the open position, the consumer medium 14 and the propellant medium 16 stored within the container reservoir 24 can pass through the container valve 26. In this embodiment, when the container valve 26 is in the open position, the consumer medium 14 and the propellant medium 16 can be dispensed from the container 12. In this embodiment, to dispense from the container 12, the consumer medium 14 and the propellant medium 16 pass from the container reservoir 24, through the container dip tube 28, then through the container valve 26, and then through the container nozzle 30. In this embodiment, the propellant medium 16 provides the force that causes the consumer medium 14 and the propellant medium 16 to dispense from the container 12 when the container valve 26 is in the open position. In this embodiment, the consumer medium 14 and the propellant medium 16 mix with one another as they dispense from the container 12. That mixture is referred to hereinafter as the consumer mixture 32. FIG. 2 illustrates an amount of the consumer mixture 32 after it has been dispensed into the hand of a consumer. In some embodiments, the consumer medium 14 and the propellant medium 16 additionally or alternatively mix to form the consumer mixture 32 before and/or after being dispensed from the container 12. In the embodiment illustrated in FIG. 1, the components of the container 12 are made of known plastic materials. In other embodiments, the components of the container 12 can be made of various other types of known materials, including, for example, aluminum and steel.

The consumer medium 14 can be a personal care medium (e.g., a shaving aid medium, a body wash medium, a sunscreen medium, etc.), or any other type of medium that is capable of being stored within the container 12 with the propellant medium 16, and that is capable of being dispensed from the container 12. The consumer medium 14 can be in one or more fluidic forms (e.g., in the form of a liquid, in the form of a gel, etc.). In some embodiments, the packaged product 10 additionally includes one or more non-fluidic additives (e.g., beads, microcapsules) that are dispersed within the consumer medium 14. In some embodiments, the consumer medium 14 has foaming and/or post-foaming characteristics. In some embodiments, the consumer medium 14 can be water-based. In some embodiments, the consumer medium 14 can be at least substantially free of solvents. The ingredients of the consumer medium 14, and/or the relative amount of each ingredient can vary. In some embodiments, the ingredients and/or the relative amounts of the ingredients in the consumer medium 14 can be selected so that the consumer medium 14 and/or the consumer mixture 32 has one or more desired characteristics (e.g., creaminess, cushion, lubricity, hydration, etc.). In the embodiment illustrated in FIG. 1, the consumer medium 14 is a shaving aid medium that includes the following ingredients:

TABLE 1

| | Percent weight by |
|---|---|
| | volume |
| Deionized water | 66.25 |
| Palmitic acid | 10.00 |
| Triethanolamine | 8.40 |
| Polyvinylpyrrolidone K-90 | 0.60 |
| Stearic acid | 3.50 |
| Sunflower oil | 5.00 |
| monoglyceride | |
| Sorbitol 70%, USP | 3.00 |
| Polyox ® WSR 301 | 0.25 |
| Aloe vera powder | 0.20 |
| Vitamin E acetate USP | 0.30 |
| Glycerine, USP | 2.00 |
| Fragrance | 0.50 |
| Total | 100.00 |

In another embodiment, the consumer medium 14 is a shaving aid medium that includes the following ingredients:

TABLE 2

| | Percent weight by |
|---|---|
| | volume |
| Deionized water | 65.90 |
| Palmitic acid | 10.00 |
| Triethanolamine | 8.50 |
| Glycerine, USP | 3.00 |
| Stearic acid | 3.50 |
| Sunflower oil monoglyceride | 5.00 |
| Sorbitol 70%, USP | 3.00 |
| Colloidal Oatmeal | 0.30 |
| Hydroxyethyl Cellulose | 0.30 |
| DL-panthenol | 0.50 |
| Total | 100.00 |

In another embodiment, the consumer medium 14 is a shaving aid medium that includes the following ingredients:

TABLE 3

| | Percent weight by |
|---|---|
| | volume |
| Water, Deionized | 64.70 |
| Palmitic acid | 2.00 |
| Triethanolamine | 2.50 |
| Glycerine, USP | 3.00 |
| Stearic acid | 1.00 |
| Sunflower oil monoglyceride | 3.00 |
| Potassium N-cocoyl glycinate | 10.00 |
| cocamidopropyl betaine | 5.00 |
| Cocamide MEA | 2.00 |
| Sorbitol 70%, USP | 3.00 |
| Aloe Vera Powder | 0.10 |
| Vitamin E Acetate | 0.10 |
| DL-Panthenol | 0.50 |
| Colloidal Oatmeal | 0.30 |
| Hydroxyethyl Cellulose | 0.30 |
| Lubragel oil | 1.50 |
| Bisabolol | 0.50 |
| Fragrance | 0.50 |
| Total | 100.00 |

In another embodiment, the consumer medium 14 is a body wash medium that includes the following ingredients:

TABLE 4

| | Percent weight by |
|---|---|
| | volume |
| Water, Deionized | 64.68 |
| Palmitic acid | 2.00 |
| Triethanolamine | 2.50 |
| Glycerine, USP | 3.00 |
| Stearic acid | 1.00 |
| Sunflower oil monoglyceride | 3.00 |
| Potassium N-cocoyl glycinate | 10.00 |
| cocamidopropyl betaine | 5.00 |
| Cocamide MEA | 2.00 |
| Sorbitol 70%, USP | 3.00 |
| Aloe Vera Powder | 0.10 |
| Vitamin E Acetate | 0.10 |
| DL-Panthenol | 0.50 |
| Colloidal Oatmeal | 0.30 |
| Jaguar C-162 | 0.30 |
| Citric Acid | 0.02 |
| Lubragel oil | 1.50 |
| Bisabolol | 0.50 |
| Fragrance | 0.50 |
| Total | 100.00 |

The propellant medium 16 includes a hydrocarbon agent, and one or more of an organoflourine agent, air, and carbon dioxide.

The hydrocarbon agent included in the propellant medium 16 can have various ingredients and various relative amounts of each ingredient. In some embodiments, the hydrocarbon agent can include: normal pentane, isopentane, normal butane, isobutane, propane, and mixtures thereof.

In embodiments in which the propellant medium 16 includes an organoflourine agent, the organoflourine agent can have various ingredients and various relative amounts of each ingredient. In some embodiments, the organoflourine agent can include: one or more difluoroethane compounds, one or more trifluoropropene compounds, one or more tetrafluoropropene compounds, one or more pentafluoropropane compounds, and mixtures thereof. Examples of acceptable difluoroethane compounds include: 1,1 difluoroethane; and ethylidene fluoride. Examples of acceptable trifluoropropene compounds include: trans-1-chloro-3,3,3-trifluoropropene. Examples of acceptable tetrafluoropropene compounds include: 2,3,3,3, tetrafluoropropene; and 1,3,3,3 tetrafluoropropene. Examples of acceptable pentafluoropropane compounds include: 1,3-dichloro-1,1,2,2,3-petafluoropropane; 1,1,1,2,2 pentafluoropropane; and 1,1,1,3,3 Pentafluoropropane.

The term “air” is used herein to refer to the mixture of gases that form Earth's atmosphere. In embodiments in which the propellant medium 16 includes air, the air thus includes nitrogen, oxygen, and trace amounts of other gases.

In embodiments in which the propellant medium 16 includes air and carbon dioxide, the carbon dioxide is in addition to any trace amounts of carbon dioxide that may already be present in the air. That is, in embodiments of the propellant medium 16 described herein as including both air and carbon dioxide, the carbon dioxide is provided in addition to the air, and in an amount that that is significantly greater than any amount of carbon dioxide that may already be present in the air.

The ingredients and/or the relative amounts of the ingredients in the propellant medium 16 can vary. In some embodiments, the ingredients and/or the relative amounts of the ingredients in the propellant medium 16 can be selected so that the propellant medium 16 and/or the consumer mixture 32 has one or more desired characteristics (e.g., creaminess, cushion, lubricity, hydration, etc.). In the embodiment illustrated in FIG. 1, the propellant medium 16 includes the following ingredients:

TABLE 5

| | Percent weight |
|---|---|
| | by volume |
| Hydrocarbon agent | |
| Isopentane | 50.00 |
| Organoflourine agent | |
| Tetrafluoropropene | 50.00 |
| Total | 100.00 |

In another embodiment, the propellant medium 16 includes the following ingredients:

TABLE 6

| | Percent weight |
|---|---|
| | by volume |
| Carbon dioxide | 20.00 |
| Hydrocarbon agent | |
| Isopentane | 56.00 |
| Isobutane | 24.00 |
| Total | 100.00 |

In another embodiment, the propellant medium 16 includes the following ingredients:

TABLE 7

| | Percent weight |
|---|---|
| | by volume |
| Hydrocarbon agent | |
| Isobutane | 20.00 |
| Organoflourine agent | |
| Pentafluoropropane | 80.00 |
| Total | 100.00 |

In another embodiment, the propellant medium 16 includes the following ingredients:

TABLE 8

| | Percent weight |
|---|---|
| | by volume |
| Carbon dioxide | 20.00 |
| Hydrocarbon agent | |
| Isopentane | 56.00 |
| Isobutane | 24.00 |
| Total | 100.00 |

In some embodiments, the desired characteristic of the propellant medium 16 and/or the consumer mixture 32 is achieved at least in part because the propellant medium 16 has a particular vapor pressure at a particular temperature, before and/or after the consumer mixture 32 is dispensed from the container 12. In the propellant medium 16 embodiment described in Table 5, the propellant medium 16 has a vapor pressure of approximately seventeen and three tenths (17.3) psi gauge (i.e., approximately one hundred nineteen (119) KPa gauge) at approximately twenty one (21) degrees Celsius (i.e., approximately seventy (70) degrees Fahrenheit). In the propellant medium 16 embodiment described in Table 6, the propellant medium 16 has a vapor pressure of approximately sixteen (16) psi gauge (i.e., approximately one hundred ten and three tenths (110.3) KPa gauge) at approximately twenty one (21) degrees Celsius (i.e., approximately seventy (70) degrees Fahrenheit). In the propellant medium 16 embodiment described in Table 7, the propellant medium 16 has a vapor pressure of approximately twenty three (23) psi gauge (i.e., approximately one hundred fifty nine (159) KPa gauge) at approximately twenty one (21) degrees Celsius (i.e., approximately seventy (70) degrees Fahrenheit). In the propellant medium 16 embodiment described in Table 8, the propellant medium 16 has a vapor pressure of approximately sixteen (16) psi gauge (i.e., approximately one hundred ten and three tenths (110.3) KPa gauge) at approximately twenty one (21) degrees Celsius (i.e., approximately seventy (70) degrees Fahrenheit).

In some embodiments in which the propellant medium 16 includes carbon dioxide, the desired characteristic of the propellant medium 16 and/or the consumer mixture 32 is achieved at least in part because the carbon dioxide is included. In the propellant medium 16 embodiments described in Tables 6 and 8, for example, the inclusion of the carbon dioxide increases the vapor pressure of the propellant medium 16, which in turn increases the creaminess and cushion of the consumer mixture 32. If the propellant medium 16 embodiments described in Tables 6 and 8 excluded the carbon dioxide, the vapor pressure of the propellant medium 16 would be only eight and nine tenths (8.9) psi gauge (i.e., approximately sixty one and four tenths (61.4) KPa gauge) at approximately twenty one (21) degrees Celsius (i.e., approximately seventy (70) degrees Fahrenheit).

The relative amounts of the consumer medium 14 and the propellant medium 16 can vary. In some embodiments, the relative amounts of the consumer medium 14 and the propellant medium 16 can be selected so that the consumer mixture 32 has one or more desired characteristics (e.g., creaminess, cushion, lubricity, hydration, etc.). In the embodiment illustrated in FIG. 1, the packaged product 10 includes ninety-four (94) percent weight by volume of the consumer medium 14 described in Table 1, and six (6) percent weight by volume of the propellant medium 16 described in Table 5. In another embodiment, the packaged product 10 includes ninety five (95) percent weight by volume of the consumer medium 14 described in Table 2, and five (5) percent weight by volume of the propellant medium 16 described in Table 6. In another embodiment, the packaged product 10 includes ninety four (94) percent weight by volume of the consumer medium 14 described in Table 3, and six (6) percent weight by volume of the propellant medium 16 described in Table 7. In another embodiment, the packaged product 10 includes ninety five (95) percent weight by volume of the consumer medium 14 described in Table 4, and five (5) percent weight by volume of the propellant medium 16 described in Table 8.

While several embodiments have been disclosed, it will be apparent to those of ordinary skill in the art that aspects of the present invention include many more embodiments and implementations. Accordingly, aspects of the present invention are not to be restricted except in light of the attached claims and their equivalents. It will also be apparent to those of ordinary skill in the art that variations and modifications can be made without departing from the true scope of the present disclosure. For example, in some instances, one or more features disclosed in connection with one embodiment can be used alone or in combination with one or more features of one or more other embodiments.

What is claimed is:

1. A packaged product, comprising:
a container with a consumer medium and a propellant medium stored therein;
wherein the container is a non-barrier type aerosol container;
wherein the propellant medium includes a hydrocarbon agent.

2. The packaged product of claim 1, wherein the container does not include a barrier that fluidly separates the consumer medium from the propellant medium.

3. The packaged product of claim 1, wherein the container includes a container reservoir and a container valve;
wherein the consumer medium and the propellant medium are stored within the container reservoir;
wherein the container valve is selectively actuatable between a closed position and an open position;
wherein the consumer medium and the propellant medium stored within the container reservoir cannot pass through the container valve when the container valve is in the closed position; and
wherein the consumer medium and the propellant medium stored within the container reservoir can pass through the container valve when the container valve is in the open position.

4. The packaged product of claim 3, wherein the volume of the reservoir is between approximately 60 ml and 350 ml.

5. The packaged product of claim 3, wherein the propellant medium provides a force that causes the consumer medium and the propellant medium to dispense from the container when the container valve is in the open position.

6. The packaged product of claim 1, wherein the consumer medium and the propellant medium are operable to mix with one another to form a consumer mixture as they dispense from the container.

7. The packaged product of claim 1, wherein the consumer medium is one of a shaving aid medium, a body wash medium, and a sunscreen medium.

8. The packaged product of claim 1, wherein the consumer medium is water-based.

9. The packaged product of claim 1, wherein the consumer medium is at least substantially free of solvents.

10. The packaged product of claim 1, wherein the hydrocarbon agent includes at least one of normal pentane, isopentane, normal butane, isobutane, and propane.

11. The packaged product of claim 1, wherein the propellant medium additionally include an organoflourine agent.

12. The packaged product of claim 11, wherein the organoflourine agent includes at least one of a difluoroethane compound, a trifluoropropene compound, a tetrafluoropropene compound, and a pentafluoropropane compound.

13. The packaged product of claim 1, wherein the propellant medium additionally includes carbon dioxide.

14. The packaged product of claim 13, wherein the propellant medium additionally includes air, and wherein the carbon dioxide is provide in an amount that that is significantly greater than any amount of carbon dioxide already present in the air.

15. The packaged product of claim 13, wherein the propellant medium includes approximately twenty percent weight by volume of carbon dioxide.

16. The packaged product of claim 6, wherein a desired characteristic of the consumer mixture is achieved at least in part because the propellant medium has a particular vapor pressure at a particular temperature.

17. The packaged product of claim 17, wherein the propellant medium additionally includes carbon dioxide, and wherein the desired characteristic of the consumer mixture is achieved at least in part because the carbon dioxide is included.

18. The packaged product of claim 16, wherein the desired characteristic is one of creaminess, cushion, lubricity, and hydration.

19. A packaged product, comprising:
a container with a consumer medium and a propellant medium stored therein;
wherein the container is a non-barrier type aerosol container;
wherein the consumer medium is water-based and at least substantially free of solvents;
wherein the propellant medium includes a hydrocarbon agent and an organoflourine agent.

20. A packaged product, comprising:
a container with a consumer medium and a propellant medium stored therein;
wherein the container is a non-barrier type aerosol container;
wherein the consumer medium is water-based and at least substantially free of solvents;
wherein the propellant medium includes a hydrocarbon agent and carbon dioxide.
